(19) United States
(12) Patent Application Publication
(10) Pub. No.: US 2026/0276616 A1
(43) Pub. Date: Sep. 17, 2026

(54) REFINED CALCULATION METHOD FOR TOTAL POLLUTANT DISCHARGE UNDER COMPLEX RIVER CONDITION

(71) Applicant: Environmental Development Center of the Ministry of Ecology and Environment, Beijing (CN)

(72) Inventors: Zhuqing WEN, Beijing (CN); Dongyang WEI, Beijing (CN); Dan SHAN, Beijing (CN); Yue REN, Beijing (CN); Huatang REN, Beijing (CN); Wenling WANG, Beijing (CN); Shujie QIAN, Beijing (CN); Ziyue SONG, Beijing (CN)

(73) Assignee: Environmental Development Center of the Ministry of Ecology and Environment, Beijing (CN)

(21) Appl. No.: 19/562,898

(22) Filed: Mar. 11, 2026

(30) Foreign Application Priority Data
Mar. 17, 2025 (CN) 202510311757.0

(51) Int. Cl. G01N 33/18; G06F 17/10

(52) U.S. Cl. CPC G01N 33/18; G06F 17/10

(57) ABSTRACT

A refined calculation method for total pollutant discharge under a complex river condition is provided. The method includes: establishing a physical experimental model, and setting a pollution load of each river reach in the physical experimental model; establishing a mathematical model of a complex river water environment based on the set physical experimental model, and calculating a pollutant concentration in a studied river reach under a preset environmental condition; and separately calculating a water environmental capacity for a pollutant in the studied river reach during a low-flow period and a high-flow period, assigning low-flow period and high-flow period weights to the studied river reach, and calculating and obtaining total permitted pollutant discharge for a watershed of the studied river reach. The method achieves accurate calculation of total permitted discharge of a characteristic pollutant under a complex river condition.

### CROSS REFERENCE TO THE RELATED APPLICATIONS

This application is based upon and claims priority to Chinese Patent Application No. 202510311757.0, filed on Mar. 17, 2025, the entire contents of which are incorporated herein by reference.

### TECHNICAL FIELD

The present invention relates to the technical field of pollutant discharge control, and more specifically, to a refined calculation method for total pollutant discharge under a complex river condition.

### BACKGROUND

In the field of river pollution control and management, accurately calculating total permitted discharge of a characteristic pollutant for a watershed is crucial for protecting river water environmental quality. Although certain achievements have been made in a calculation method for total river pollutant discharge, the existing technology still has many deficiencies when facing a complex river condition. First, existing research has insufficient understanding of a dispersion characteristic of a complex river. Complex river channel morphology, large seasonal variations in water volume, high water and sediment content, and coexistence of frozen and non-frozen reaches make the transport and mixing mechanism of pollutants in a river extremely complex. However, the existing technology has difficulty in comprehensively and accurately grasping an impact of these factors on pollutant transport and mixing, and cannot precisely determine a transverse dispersion coefficient and a longitudinal dispersion coefficient, resulting in significant errors in calculating a pollutant diffusion range and a concentration change.

Further, the existing model has limitations in constructing a mathematical model of a river water environment. In a conventional two-dimensional water environment mathematical model, determining methods for a longitudinal dispersion coefficient, a transverse dispersion coefficient, and a pollutant attenuation coefficient are simple, failing to fully consider comprehensive influence of various factors under a complex river condition. For example, when calculating the longitudinal dispersion coefficient, key factors such as a river flow characteristic, a friction resistance characteristic, and cross-sectional dimensions are not comprehensively covered; calculation of the transverse dispersion coefficient often ignores complex correlation with the longitudinal dispersion coefficient; and for the pollutant attenuation coefficient, an impact of special conditions such as high sediment content and freezing on a water self-purification capacity is not fully considered, resulting in significant deviation between a model calculation result and an actual condition, making it difficult to provide a reliable basis for river pollution control.

Therefore, how to calculate total permitted discharge of a characteristic pollutant under a complex river condition is an urgent problem that needs to be solved by those skilled in the art.

### SUMMARY

In view of this, the present invention provides a refined calculation method for total pollutant discharge under a complex river condition, to solve the problems existing in the above-mentioned background art.

To achieve the above objectives, the present invention adopts the following technical solutions.

A refined calculation method for total pollutant discharge under a complex river condition, including:
establishing a physical experimental model, and setting a pollution load of each river reach in the physical experimental model;
establishing a mathematical model of a complex river water environment based on the set physical experimental model, and calculating a pollutant concentration in a studied river reach under a preset environmental condition; and
separately calculating a water environmental capacity for a pollutant in the studied river reach during a low-flow period and a high-flow period, assigning low-flow period and high-flow period weights to the studied river reach, and calculating and obtaining total permitted pollutant discharge for a watershed of the studied river reach.

Preferably, the preset environmental condition includes:
a first preset environmental condition: a complex channel morphology;
a second preset environmental condition: a complex channel morphology and a high sediment condition;
a third preset environmental condition: a complex channel morphology and a frozen condition;
a fourth preset environmental condition: a complex channel morphology and a high sediment and frozen condition; and
any one of the preset environmental conditions.

Preferably, calculating a pollutant concentration in the studied river reach under the first preset environmental condition specifically includes:

$$\frac{\partial ( H C )}{\partial t} + \frac{\partial ( H u C )}{\partial x} + \frac{\partial ( H v C )}{\partial y} = \frac{\partial }{\partial x} [ H \times {m ( \frac{W}{H} )}^{d} {( \frac{{u}^{′}}{{u}_{*}} )}^{i} H {u}_{*} \times \frac{\partial C}{\partial x} ] + \frac{\partial }{\partial y} \{ H \{ a [ \frac{{( Q / H )}^{( 1 + γ )} {( W / H )}^{( 1 - γ )}}{{D}_{x}^{γ}} ] + b \} \frac{\partial C}{\partial y} \} + F ( k ) C - S = 0 ;$$

$$F ( k ) = F ( C , DO , Q , t , Δ x , Δ y ) ;$$

Herein, His a water depth; W is a river width; a, b, γ, m, d, and i are constants; u and v are velocity components in an x direction and a y direction respectively; u′ is a cross-sectional average flow velocity, and u_{*} is a bed bottom friction velocity; [symbol] is water discharge; Dₓ is a river longitudinal dispersion coefficient; F(k) is a pollutant attenuation coefficient under the first preset environmental condition; C is a pollutant concentration; S is an external source; DO is dissolved oxygen content in water; [symbol] is water discharge; t is time; and Δx and Δy are a longitudinal transport distance and a transverse transport distance respectively.

Preferably, calculating a pollutant concentration of the studied river reach under the second preset environmental condition specifically includes:

$$\frac{\partial ( H C )}{\partial t} + \frac{\partial ( H u C )}{\partial x} + \frac{\partial ( H v C )}{\partial y} = \frac{\partial }{\partial x} [ H \times {m ( \frac{W}{H} )}^{d} {( \frac{{u}^{′}}{{u}_{*}} )}^{i} H {u}_{*} \times \frac{\partial C}{\partial x} ] + \frac{\partial }{\partial y} \{ H \{ a [ \frac{{( Q / H )}^{( 1 + γ )} {( W / H )}^{( 1 - γ )}}{{D}_{x}^{γ}} ] + b \} \frac{\partial C}{\partial y} \} + {F ( k )}_{s} C - S = 0 ;$$

$${F ( k )}_{s} = F ( C , {DO}_{s} , Q , t , Δ x , Δ y ) ;$$

$${DO}_{s} = {a}_{1} - {b}_{1} T - c ρ ;$$

Herein, His a water depth; W is a river width; a, b, γ, m, d, and i are constants; u and v are velocity components in an x direction and a y direction respectively; u′ is a cross-sectional average flow velocity, and u_{*} is a bed bottom friction velocity; [symbol] is water discharge; Dₓ is a river longitudinal dispersion coefficient; F(k)ₛ is a pollutant attenuation coefficient under the second preset environmental condition; C is a pollutant concentration; S is an external source; DOₛ represents an impact of water-sediment content on dissolved oxygen; T represents a water temperature, ρ represents a sediment concentration, a₁, b₁, and c are coefficients; [symbol] is water discharge; t is time; and Δx and Δy are a longitudinal transport distance and a transverse transport distance respectively.

Preferably, calculating a pollutant concentration of the studied river reach under the third preset environmental condition specifically includes:

$$\frac{\partial ( H C )}{\partial t} + \frac{\partial ( H u C )}{\partial x} + \frac{\partial ( H v C )}{\partial y} = \frac{\partial }{\partial x} [ H \times {m ( \frac{W}{H} )}^{d} {( \frac{{u}^{′}}{{u}_{*}} )}^{i} H {u}_{*} \times \frac{\partial C}{\partial x} ] + \frac{\partial }{\partial y} \{ H \{ a [ \frac{{( Q / H )}^{( 1 + γ )} {( W / H )}^{( 1 - γ )}}{{D}_{x}^{γ}} ] + b \} \frac{\partial C}{\partial y} \} + {F ( k )}_{i} C - S = 0 ;$$

$${F ( k )}_{i} = F ( C , {DO}_{i} , Q , t , Δ x , Δ y ) ;$$

$${DO}_{i} = {eh}^{2} + fh + g ;$$

Herein, H is a water depth; W is a river width; a, b, γ, m, d, and i are constants; u and v are velocity components in an x direction and a y direction respectively; u′ is a cross-sectional average velocity, and u_{*} is a bed bottom friction velocity; [symbol] is water discharge; Dₓ is a longitudinal dispersion coefficient of the river; F(k)ᵢ is a pollutant attenuation coefficient under the third preset environmental condition; C is a pollutant concentration; S is an external source; DOᵢ represents an impact of freezing on dissolved oxygen content in water; h represents an ice layer thickness, and e, f, and g are coefficients; [symbol] is water discharge; t is time; and Δx and Δy are a longitudinal transport distance and a transverse transport distance respectively.

Preferably, calculating a pollutant concentration of the studied river reach under the fourth preset environmental condition specifically includes:

$$\frac{\partial ( H C )}{\partial t} + \frac{\partial ( H u C )}{\partial x} + \frac{\partial ( H v C )}{\partial y} = \frac{\partial }{\partial x} [ H \times {m ( \frac{W}{H} )}^{d} {( \frac{{u}^{′}}{{u}_{*}} )}^{i} H {u}_{*} \times \frac{\partial C}{\partial x} ] + \frac{\partial }{\partial y} \{ H \{ a [ \frac{{( Q / H )}^{( 1 + γ )} {( W / H )}^{( 1 - γ )}}{{D}_{x}^{γ}} ] + b \} \frac{\partial C}{\partial y} \} + {F ( k )}_{s , i} C - S = 0 ;$$

$${F ( k )}_{s , i} = F ( C , {DO}_{s} , {DO}_{i} , Q , t , Δ x , Δ y ) ;$$

Herein, F(k)_{s,i} is a pollutant attenuation coefficient under the fourth preset environmental condition.

Preferably, that the pollutant water environmental capacity of the studied river reach during a low-flow period and a high-flow period is calculated separately is represented by the formula:

$$w = {Q}_{0} ( {C}_{s} - {C}_{0} ) + {KVC}_{s} ;$$

Herein, Q₀ is inflow discharge at an inlet cross-section, Cₛ is a water quality standard for the water body, C₀ is a water quality concentration at the inlet cross-section, K is a pollutant attenuation coefficient, and V is a water body volume.

According to the above technical solutions, compared with the existing technology, the present invention discloses a refined calculation method for total pollutant discharge under a complex river condition. Through physical experiments, transport and mixing mechanisms of a pollutant in a complex river channel morphology model are measured and analyzed, and impacts of different cross-sectional morphologies, hydrodynamic condition changes, water sediment content, and freezing on a flow velocity and pollutant concentration changes in a river channel are measured, thereby determining influencing factors of transverse and longitudinal dispersion coefficients, analyzing and revealing transport and mixing mechanisms of the pollutant under the complex river condition, establishing calculation formulas for transverse and longitudinal dispersion coefficients for a river watershed comprehensively considering diverse channel morphologies, large seasonal variations in a water volume, high water-sediment content, and coexistence of frozen and non-frozen reaches, to achieve accurate calculation of total permitted discharge of a characteristic pollutant under a complex river condition.

### BRIEF DESCRIPTION OF THE DRAWINGS

To more clearly illustrate the technical solutions in embodiments of the present invention or in the conventional technology, the drawings that need to be used in the descriptions of embodiments or the conventional technology will be briefly introduced below. Obviously, the drawings in the following description are only embodiments of the present invention. For those of ordinary skill in the art, other drawings can be obtained based on the provided drawings without creative efforts.

FIG. 1 is a flowchart of method steps provided by the present invention;

FIG. 2 is a schematic diagram of a planar morphology of a physical model provided by the present invention; and

FIG. 3 is a schematic diagram of a cross-sectional morphology of a physical model provided by the present invention.

### DETAILED DESCRIPTION OF THE EMBODIMENTS

The technical solutions in embodiments of the present invention will be clearly and completely described below in conjunction with the drawings in embodiments of the present invention. Obviously, the described embodiments are only some embodiments of the present invention instead of all embodiments. Based on embodiments of the present invention, all other embodiments obtained by those of ordinary skill in the art without creative effort fall within the scope of protection of the present invention.

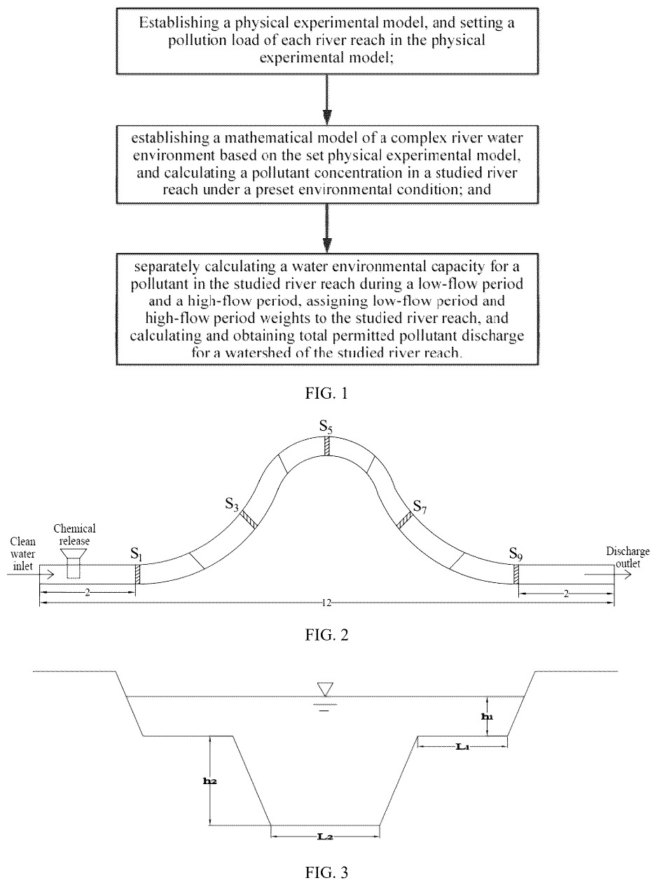

An embodiment of the present invention discloses a refined calculation method for total pollutant discharge under a complex river condition. As shown in FIG. 1, the method includes:
establishing a physical experimental model, and setting a pollution load of each river reach of the physical experimental model;
establishing a mathematical model of a complex river water environment based on the set physical experimental model, and calculating a pollutant concentration in a studied river reach under a preset environmental condition; and
separately calculating a water environmental capacity for a pollutant in the studied river reach during a low-flow period and a high-flow period, assigning low-flow period and high-flow period weights to the studied river reach, and calculating and obtaining total permitted pollutant discharge for a watershed of the studied river reach.

In a specific embodiment, the preset environmental condition includes:
a first preset environmental condition: a complex channel morphology;
a second preset environmental condition: a complex channel morphology and a high sediment condition;
a third preset environmental condition: a complex channel morphology and a frozen condition;
a fourth preset environmental condition: a complex channel morphology and a high sediment and frozen condition; and
any one of the preset environmental conditions.

Herein, the complex river channel generally refers to a river where reaches have different channel cross-sectional morphologies, including bends, straight reaches, constrictions, and bifurcations. Different channel morphologies have different impacts on pollutant transport, which are difficult to accurately simulate using a simple straight river model. The high sediment condition refers to a river with high sediment content, generally referring to a river with high suspended solids (suspended sediment) content, fine suspended solids, a shallow riverbed depth, and a slow flow velocity. Among them, the suspended solids content in the water body exceeds 10 mg/L, of which suspended solids with a particle size of 4 mm or more exceed 2 mg/L.

In a specific embodiment, calculating a pollutant concentration in the studied river reach under the first preset environmental condition specifically includes:

$$\frac{\partial ( H C )}{\partial t} + \frac{\partial ( H u C )}{\partial x} + \frac{\partial ( H v C )}{\partial y} = \frac{\partial }{\partial x} [ H \times {m ( \frac{W}{H} )}^{d} {( \frac{{u}^{′}}{{u}_{*}} )}^{i} H {u}_{*} \times \frac{\partial C}{\partial x} ] + \frac{\partial }{\partial y} \{ H \{ a [ \frac{{( Q / H )}^{( 1 + γ )} {( W / H )}^{( 1 - γ )}}{{D}_{x}^{γ}} ] + b \} \frac{\partial C}{\partial y} \} + F ( k ) C - S = 0 ;$$

$$F ( k ) = F ( C , DO , Q , t , Δ x , Δ y ) ;$$

Herein, His a water depth; W is a river width; a, b, γ, m, d, and i are constants; u and v are velocity components in an x direction and a y direction respectively; u′ is a cross-sectional average flow velocity, and u_{*} is a bed bottom friction velocity; [symbol] is water discharge; Dₓ is a river longitudinal dispersion coefficient; F(k) is a pollutant attenuation coefficient under the first preset environmental condition; C is a pollutant concentration; S is an external source; DO is dissolved oxygen content in water; [symbol] is water discharge; t is time; and Δx and Δy are a longitudinal transport distance and a transverse transport distance respectively.

In a specific embodiment, calculating a pollutant concentration of the studied river reach under the second preset environmental condition specifically includes:

$$\frac{\partial ( H C )}{\partial t} + \frac{\partial ( H u C )}{\partial x} + \frac{\partial ( H v C )}{\partial y} = \frac{\partial }{\partial x} [ H \times {m ( \frac{W}{H} )}^{d} {( \frac{{u}^{′}}{{u}_{*}} )}^{i} H {u}_{*} \times \frac{\partial C}{\partial x} ] + \frac{\partial }{\partial y} \{ H \{ a [ \frac{{( Q / H )}^{( 1 + γ )} {( W / H )}^{( 1 - γ )}}{{D}_{x}^{γ}} ] + b \} \frac{\partial C}{\partial y} \} + {F ( k )}_{s} C - S = 0 ;$$

$${F ( k )}_{s} = F ( C , {DO}_{s} , Q , t , Δ x , Δ y ) ;$$

$${DO}_{s} = {a}_{1} - {b}_{1} T - c ρ ;$$

Herein, H is a water depth; W is a river width; a, b, γ, m, d, and i are constants; u and v are velocity components in an x direction and a y direction respectively; u′ is a cross-sectional average flow velocity, and u_{*} is a bed bottom friction velocity; [symbol] is water discharge; Dₓ is a river longitudinal dispersion coefficient; F(k), is a pollutant attenuation coefficient under the second preset environmental condition; C is a pollutant concentration; S is an external source; DOₛ represents an impact of water-sediment content on dissolved oxygen; T represents a water temperature, ρ represents a sediment concentration, a₁, b₁, and c are coefficients; [symbol] is water discharge; t is time; and Δx and Δy are a longitudinal transport distance and a transverse transport distance respectively.

In a specific embodiment, calculating a pollutant concentration of the studied river reach under the third preset environmental condition specifically includes:

$$\frac{\partial ( H C )}{\partial t} + \frac{\partial ( H u C )}{\partial x} + \frac{\partial ( H v C )}{\partial y} = \frac{\partial }{\partial x} [ H \times {m ( \frac{W}{H} )}^{d} {( \frac{{u}^{′}}{{u}_{*}} )}^{i} H {u}_{*} \times \frac{\partial C}{\partial x} ] + \frac{\partial }{\partial y} \{ H \{ a [ \frac{{( Q / H )}^{( 1 + γ )} {( W / H )}^{( 1 - γ )}}{{D}_{x}^{γ}} ] + b \} \frac{\partial C}{\partial y} \} + {F ( k )}_{i} C - S = 0 ;$$

$${F ( k )}_{i} = F ( C , {DO}_{i} , Q , t , Δ x , Δ y ) ;$$

$${DO}_{i} = {eh}^{2} + fh + g ;$$

Herein, His a water depth; W is a river width; a, b, γ, m, d, and i are constants; u and v are velocity components in an x direction and a y direction respectively; u′ is a cross-sectional average velocity, and u_{*} is a bed bottom friction velocity; [symbol] is water discharge; Dₓ is a longitudinal dispersion coefficient of the river; F(k)ᵢ is a pollutant attenuation coefficient under the third preset environmental condition; C is a pollutant concentration; S is an external source; DOᵢ represents an impact of freezing on dissolved oxygen content in water; h represents an ice layer thickness, and e, f, and g are coefficients; [symbol] is water discharge; t is time; and Δx and Δy are a longitudinal transport distance and a transverse transport distance respectively.

In a specific embodiment, calculating a pollutant concentration of the studied river reach under the fourth preset environmental condition specifically includes:

$$\frac{\partial ( H C )}{\partial t} + \frac{\partial ( H u C )}{\partial x} + \frac{\partial ( H v C )}{\partial y} = \frac{\partial }{\partial x} [ H \times {m ( \frac{W}{H} )}^{d} {( \frac{{u}^{′}}{{u}_{*}} )}^{i} H {u}_{*} \times \frac{\partial C}{\partial x} ] + \frac{\partial }{\partial y} \{ H \{ a [ \frac{{( Q / H )}^{( 1 + γ )} {( W / H )}^{( 1 - γ )}}{{D}_{x}^{γ}} ] + b \} \frac{\partial C}{\partial y} \} + {F ( k )}_{s , i} C - S = 0 ;$$

$${F ( k )}_{s , i} = F ( C , {DO}_{s} , {DO}_{i} , Q , t , Δ x , Δ y ) ;$$

Herein, F(k)_{s,i} is a pollutant attenuation coefficient under the fourth preset environmental condition.

In a specific embodiment, that the pollutant water environmental capacity of the studied river reach during a low-flow period and a high-flow period is calculated separately is represented by the formula:

$$w = {Q}_{0} ( {C}_{s} - {C}_{0} ) + {KVC}_{s} ;$$

Herein, Q₀ is inflow discharge at an inlet cross-section, Cₛ is a water quality standard for the water body, C₀ is a water quality concentration at the inlet cross-section, K is a pollutant attenuation coefficient, and V is a water body volume.

In a specific embodiment, a straight-curved-straight channel physical experimental model is constructed for complex channel morphologies of the upper and middle reaches of the Yellow River. The physical experimental model has a total length of 12 m, a maximum water surface width of 1 m, a main experimental reach length of approximately 10 m, and an adjustable bottom slope. An inflow system and an outflow system are disposed at the upstream and the downstream of the physical experimental model respectively. By adjusting a water depth in the model, a model cross-section exhibits various morphologies such as a wide-shallow type and a narrow-deep type. Two straight reaches before a bend inlet reach and after an outlet reach are both 2 m long, as shown in FIG. 2 and FIG. 3.

The physical experiment is conducted according to the following steps.
i. Select monitoring cross-sections at 45° intervals along a continuous curved reach of the physical experimental model, measure three-dimensional flow velocity distribution in the model with an ADCP measuring instrument, and release a tracer; measure a longitudinal variation of a pollutant at different positions of the cross-section by using a concentration measuring instrument, and analyze transport and mixing mechanisms of a pollutant in a complex river channel morphology model;
ii. by adjusting a water depth in a flume and changing a flume cross-sectional shape, measure impacts of different cross-sectional shapes on pollutant concentration variations;
iii. by changing an inlet flow rate, measure an impact of a hydrodynamic condition change on a variation in a flow velocity and concentration distribution within a channel;
iv. release different amounts of sediment, repeat step i, and analyze an impact of sediment content in a water body on the variation in the flow velocity and the pollutant concentration; and
v. based on relevant research, select an appropriate plywood for floating on a water surface and fix the plywood to a model sidewall, repeat steps i and iv to simulate an impact of freezing on the variation in the water flow velocity and the pollutant concentration; and
conduct in-depth analysis and organization of the above physical experimental data to determine influencing factors of transverse and longitudinal dispersion coefficients, analyze and reveal transport and mixing mechanisms of the pollutant under the complex river condition, and establish calculation formulas for transverse and longitudinal dispersion coefficients for the upper and middle reaches of the Yellow River that comprehensively consider diverse channel morphologies, significant seasonal variations in a water volume, high sediment content, and coexistence of frozen and non-frozen reaches.

A spatiotemporal variation process of the pollutant concentration in the water body mainly includes an advection-diffusion process of the pollutant with a water flow and a biochemical degradation and decay process of the pollutant. A mass balance equation for water quality process control in a two-dimensional planar model is typically expressed by the following pollutant advection-diffusion-decay equation:

$$\frac{\partial ( H C )}{\partial t} + \frac{\partial ( H u C )}{\partial x} + \frac{\partial ( H v C )}{\partial y} = \frac{\partial }{\partial x} ( H {D}_{x} \frac{\partial C}{\partial x} ) + \frac{\partial }{\partial y} ( H {D}_{y} \frac{\partial C}{\partial y} ) + {S}_{c} - S = 0 \qquad ( 1 )$$

Herein, H represents a water depth; C is a pollutant concentration; u and v are velocity components in the x direction and the y direction respectively; Dₓ and D_{y} are horizontal dispersion coefficients in the x direction and the y direction respectively, namely, a longitudinal dispersion coefficient and a transverse dispersion coefficient of the river; Sc is a decay term related to transported substance concentration; and S is the external source or a sink term.

$${S}_{c} = k \times C \qquad ( 2 )$$

k represents a decay rate of a pollutant in the water body due to biochemical degradation and other processes.

In calculating a river water environmental capacity based on a two-dimensional water environment mathematical model, the most important task is to determine values of Dₓ, D_{y}, and k.

In this project, the longitudinal dispersion coefficient Dₓ of the river is calculated using an empirical formula based on a river flow characteristic, a friction resistance characteristic, cross-sectional dimensions, and the like:

$${D}_{x} = {m ( \frac{W}{H} )}^{d} {( \frac{{u}^{′}}{{u}_{*}} )}^{i} H {u}_{*} \qquad ( 3 )$$

Herein, Wis a river width; His a water depth; u′ is a cross-sectional average velocity; and u_{*} is a bed bottom friction velocity. The constants m, d, and i are calculated based on physical experiments.

For the transverse dispersion coefficient D_{y}, since a transverse flow velocity in a channel is generally much smaller than a longitudinal flow velocity under normal conditions, an impact of transverse dispersion is relatively small. The present invention solves the transverse dispersion coefficient through the longitudinal dispersion coefficient based on an empirical formula:

$${D}_{y} = a [ \frac{{( Q / H )}^{( 1 + γ )} {( W / H )}^{( 1 - γ )}}{{D}_{x}^{γ}} ] + b \qquad ( 4 )$$

The pollutant attenuation coefficient k is determined by a plurality of factors. For complex conditions in the upper and middle reaches of the Yellow River, the present invention primarily considers an impact of high sediment content and coexistence of frozen and non-frozen reaches on dissolved oxygen content in the water body, namely, on a self-purification capacity of the water body, and then calibrates a solution equation for a pollutant attenuation coefficient through a method combining numerical simulation and physical modeling.

$$F ( k ) = F ( C , DO , Q , t , Δ x , Δ y ) \qquad ( 5 )$$

Herein, C is a pollutant concentration; DO is the dissolved oxygen content in the water body; [symbol] is water discharge; t is time; and Δx and Δy are a longitudinal transport distance and a transverse transport distance respectively. Thus, it can be seen that the dissolved oxygen content DO is an important variable for calculating the pollutant attenuation coefficient.

In the present invention, the dissolved oxygen content in the water body is mainly affected by water-sediment content and a frozen condition, where an impact of freezing on the self-purification capacity of the water body occurs only during a low-flow period. A relationship between the dissolved oxygen content and sediment content can be expressed as:

$${DO}_{s} = {a}_{1} - {b}_{1} T - c ρ \qquad ( 6 )$$

Herein, DOₛ represents an impact of the water-sediment content on dissolved oxygen; T represents a water temperature; p represents the sediment content; and the coefficients a₁, b₁, and c can be calculated based on physical experiments.

Therefore, considering only impacts of water and sediment, the pollutant attenuation coefficient is:

$${F ( k )}_{s} = F ( C , {DO}_{s} , Q , t , Δ x , Δ y ) \qquad ( 7 )$$

Regarding an impact of freezing on the dissolved oxygen content in the water body, the dissolved oxygen content exhibits a “U-shaped” variation curve during a frozen period. A proposed equation is:

$$D {O}_{i} = e {h}^{2} + f h + g ( e > 0 ) \qquad ( 8 )$$

Herein, DOᵢ represents the impact of freezing on the dissolved oxygen content in the water body; h represents an ice layer thickness; and coefficients e, f, and g can be calculated based on measured data from physical experiments.

Therefore, considering only the impact of freezing, the pollutant attenuation coefficient is:

$${F ( k )}_{i} = F ( C , {DO}_{i} , Q , t , Δ x , Δ y ) \qquad ( 9 )$$

Considering both the impacts of water-sediment and freezing, the pollutant attenuation coefficient is:

$${F ( k )}_{s , i} = F ( C , D {O}_{s} , {DO}_{i} , Q , t , Δ x , Δ y ) \qquad ( 10 )$$

In summary, under a complex river channel morphology, the mass balance equation for water quality process control in the two-dimensional model is improved as follows:

$$\frac{\partial ( H C )}{\partial t} + \frac{\partial ( H u C )}{\partial x} + \frac{\partial ( H v C )}{\partial y} = \frac{\partial }{\partial x} [ H \times {m ( \frac{W}{H} )}^{d} {( \frac{{u}^{′}}{{u}_{*}} )}^{i} H {u}_{*} \times \frac{\partial C}{\partial x} ] + \frac{\partial }{\partial y} \{ H \{ a [ \frac{{( Q / H )}^{( 1 + γ )} {( W / H )}^{( 1 - γ )}}{{D}_{x}^{γ}} ] + b \} \frac{\partial C}{\partial y} \} + F ( k ) C - S = 0 ; \qquad ( 11 )$$

Under a complex river channel morphology and a high sediment condition, the mass balance equation for water quality process control in the two-dimensional model is improved to:

$$\frac{\partial ( H C )}{\partial t} + \frac{\partial ( H u C )}{\partial x} + \frac{\partial ( H v C )}{\partial y} = \frac{\partial }{\partial x} [ H \times {m ( \frac{W}{H} )}^{d} {( \frac{{u}^{′}}{{u}_{*}} )}^{i} H {u}_{*} \times \frac{\partial C}{\partial x} ] + \frac{\partial }{\partial y} \{ H \{ a [ \frac{{( Q / H )}^{( 1 + γ )} {( W / H )}^{( 1 - γ )}}{{D}_{x}^{γ}} ] + b \} \frac{\partial C}{\partial y} \} + {F ( k )}_{s} C - S = 0 ; \qquad ( 12 )$$

Under a complex river channel morphology and a frozen condition, the mass balance equation for water quality process control in the two-dimensional model is improved to:

$$\frac{\partial ( H C )}{\partial t} + \frac{\partial ( H u C )}{\partial x} + \frac{\partial ( H v C )}{\partial y} = \frac{\partial }{\partial x} [ H \times {m ( \frac{W}{H} )}^{d} {( \frac{{u}^{′}}{{u}_{*}} )}^{i} H {u}_{*} \times \frac{\partial C}{\partial x} ] + \frac{\partial }{\partial y} \{ H \{ a [ \frac{{( Q / H )}^{( 1 + γ )} {( W / H )}^{( 1 - γ )}}{{D}_{x}^{γ}} ] + b \} \frac{\partial C}{\partial y} \} + {F ( k )}_{i} C - S = 0 ; \qquad ( 13 )$$

Under a complex river channel morphology, a high sediment condition, and a frozen condition, the mass balance equation for water quality process control in the two-dimensional model is improved to:

$$\frac{\partial ( H C )}{\partial t} + \frac{\partial ( H u C )}{\partial x} + \frac{\partial ( H v C )}{\partial y} = \frac{\partial }{\partial x} [ H \times {m ( \frac{W}{H} )}^{d} {( \frac{{u}^{′}}{{u}_{*}} )}^{i} H {u}_{*} \times \frac{\partial C}{\partial x} ] + \frac{\partial }{\partial y} \{ H \{ a [ \frac{{( Q / H )}^{( 1 + γ )} {( W / H )}^{( 1 - γ )}}{{D}_{x}^{γ}} ] + b \} \frac{\partial C}{\partial y} \} + {F ( k )}_{s , i} C - S = 0 ; \qquad ( 14 )$$

A pollution load in the studied watershed includes two categories: a point source and a non-point source, where non-point source pollution is primarily concentrated during a high-flow period. Therefore, from a perspective of total amount control, permitted pollutant discharge is calculated separately for a point source pollution load and a non-point source pollution load corresponding to different hydrological conditions, and the combination of both represents total permitted pollutant discharge for the watershed.

Specific calculation steps are as follows:
i. Generalize a point source pollution load to each river reach based on an actual position of a discharge outlet into a river, while simultaneously allocating non-point source pollution to river reaches surrounding a respective land area;
ii. calculate a pollutant concentration of each river reach using an established two-dimensional water environment mathematical model for a complex river;
iii. separately calculate a water environmental capacity for a pollutant in the studied river reach during a low-flow period and a high-flow period, where a calculation formula is as follows:

$$w = {Q}_{0} ( {C}_{s} - {C}_{0} ) + {KVC}_{s} \qquad ( 15 )$$

where Q₀ is an inflow rate at an inlet cross-section; Cₛ is a water quality standard for the water body; C₀ is a water quality concentration at the inlet cross-section; K is a pollutant attenuation coefficient; and V is the water body volume; and
iv. assign weights to the water environmental capacity during a low-flow period and a high-flow period based on actual conditions, comprehensively calculate a watershed water environmental capacity, and thereby determine the total permitted pollutant discharge for the watershed.

Various embodiments in this specification are described in a progressive manner, with each embodiment focusing on differences from other embodiments, and identical or similar parts among embodiments can be referenced to each other. As for the apparatus disclosed in embodiments, since it corresponds to the method disclosed in the embodiments, the descriptions are relatively simple, and relevant aspects can be referred to in the method section.

The above descriptions of the disclosed embodiments enable those skilled in the art to implement or use the present invention. Various modifications to these embodiments will be apparent to those skilled in the art, and the general principles defined herein may be implemented in other embodiments without departing from the spirit or scope of the present invention. Therefore, the present invention is not to be limited to the embodiments shown herein but is to be accorded the widest scope consistent with the principles and novel features disclosed herein.

What is claimed is:

1. A refined calculation method for total pollutant discharge under a complex river condition, comprising:
establishing a physical experimental model, and setting a pollution load of each river reach in the physical experimental model;
establishing a mathematical model of a complex river water environment based on the set physical experimental model, and calculating a pollutant concentration in a studied river reach under a preset environmental condition; and
separately calculating a water environmental capacity for a pollutant in the studied river reach during a low-flow period and a high-flow period, assigning low-flow period and high-flow period weights to the studied river reach, and calculating and obtaining total permitted pollutant discharge for a watershed of the studied river reach, wherein
the preset environmental condition comprises:
a first preset environmental condition: a complex channel morphology;
a second preset environmental condition: a complex channel morphology and a high sediment condition;
a third preset environmental condition: a complex channel morphology and a frozen condition;
a fourth preset environmental condition: a complex channel morphology and a high sediment and frozen condition; and
any one of the preset environmental conditions;
calculating a pollutant concentration in the studied river reach under the first preset environmental condition specifically comprises:
$$\frac{\partial ( HC )}{\partial t} + \frac{\partial ( HuC )}{\partial x} + \frac{\partial ( HvC )}{\partial y} = \frac{\partial }{\partial x} [ H \times m {( \frac{W}{H} )}^{d} {( \frac{{u}^{′}}{{u}_{*}} )}^{i} {Hu}_{*} \times \frac{\partial C}{\partial x} ] + \frac{\partial }{\partial y} \{ H \{ a [ \frac{{( Q / H )}^{( 1 + γ )} {( W / H )}^{( 1 - γ )}}{{D}_{x}^{γ}} ] + b \} \frac{\partial C}{\partial y} \} + F ( k ) C - S = 0 ;$$
$$F ( k ) = F ( C , DO , Q , t , Δ x , Δ y ) ;$$
wherein H is a water depth; W is a river width; a, b, γ, m, d, and i are constants; u and v are velocity components in an x direction and a y direction respectively; u′ is a cross-sectional average flow velocity, and u_{*} is a bed bottom friction velocity; [symbol] is water discharge; Dₓ is a river longitudinal dispersion coefficient; F(k) is a pollutant attenuation coefficient under the first preset environmental condition; C is a pollutant concentration; S is an external source; DO is dissolved oxygen content in water; [symbol] is water discharge; tis time; and Δx and Δy are a longitudinal transport distance and a transverse transport distance respectively;
calculating a pollutant concentration in the studied river reach under the second preset environmental condition specifically comprises:
$$\frac{\partial ( HC )}{\partial t} + \frac{\partial ( HuC )}{\partial x} + \frac{\partial ( HvC )}{\partial y} = \frac{\partial }{\partial x} [ H \times m {( \frac{W}{H} )}^{d} {( \frac{{u}^{′}}{{u}_{*}} )}^{i} {Hu}_{*} \times \frac{\partial C}{\partial x} ] + \frac{\partial }{\partial y} \{ H \{ a [ \frac{{( Q / H )}^{( 1 + γ )} {( W / H )}^{( 1 - γ )}}{{D}_{x}^{γ}} ] + b \} \frac{\partial C}{\partial y} \} + {F ( k )}_{s} C - S = 0 ;$$
$${F ( k )}_{s} = F ( C , {DO}_{s} , Q , t , Δ x , Δ y ) ;$$
$${DO}_{s} = {a}_{1} - {b}_{1} T - c ρ ;$$
wherein His a water depth; W is a river width; a, b, γ, m, d, and i are constants; u and v are velocity components in an x direction and a y direction respectively; u′ is a cross-sectional average flow velocity, and u_{*} is a bed bottom friction velocity; [symbol] is water discharge; Dₓ is a river longitudinal dispersion coefficient; F(k)ₛ is a pollutant attenuation coefficient under the second preset environmental condition; C is a pollutant concentration; S is an external source; DOₛ represents an impact of water-sediment content on dissolved oxygen; T represents a water temperature, ρ represents a sediment concentration, a₁, b₁, and c are coefficients; [symbol] is water discharge; t is time; and Δx and Δy are a longitudinal transport distance and a transverse transport distance respectively;
calculating a pollutant concentration in the studied river reach under the third preset environmental condition specifically comprises:
$$\frac{\partial ( HC )}{\partial t} + \frac{\partial ( HuC )}{\partial x} + \frac{\partial ( HvC )}{\partial y} = \frac{\partial }{\partial x} [ H \times m {( \frac{W}{H} )}^{d} {( \frac{{u}^{′}}{{u}_{*}} )}^{i} {Hu}_{*} \times \frac{\partial C}{\partial x} ] + \frac{\partial }{\partial y} \{ H \{ a [ \frac{{( Q / H )}^{( 1 + γ )} {( W / H )}^{( 1 - γ )}}{{D}_{x}^{γ}} ] + b \} \frac{\partial C}{\partial y} \} + {F ( k )}_{i} C - S = 0 ;$$
$${F ( k )}_{i} = F ( C , {DO}_{i} , Q , t , Δ x , Δ y ) ;$$
$${DO}_{i} = {eh}^{2} + fh + g ;$$
wherein H is a water depth; W is a river width; a, b, γ, m, d, and i are constants; u and v are velocity components in an x direction and a y direction respectively; u′ is a cross-sectional average velocity, and u_{*} is a bed bottom friction velocity; [symbol] is water discharge; Dₓ is a river longitudinal dispersion coefficient; F(k)ᵢ is a pollutant attenuation coefficient under the third preset environmental condition; C is a pollutant concentration; S is an external source; DOᵢ represents an impact of freezing on dissolved oxygen content in water; h represents an ice layer thickness, and e, f, and g are coefficients; [symbol] is water discharge; t is time; and Δx and Δy are a longitudinal transport distance and a transverse transport distance respectively; and
calculating a pollutant concentration in the studied river reach under the fourth preset environmental condition specifically comprises:
$$\frac{\partial ( HC )}{\partial t} + \frac{\partial ( HuC )}{\partial x} + \frac{\partial ( HvC )}{\partial y} = \frac{\partial }{\partial x} [ H \times m {( \frac{W}{H} )}^{d} {( \frac{{u}^{′}}{{u}_{*}} )}^{i} {Hu}_{*} \times \frac{\partial C}{\partial x} ] + \frac{\partial }{\partial y} \{ H \{ a [ \frac{{( Q / H )}^{( 1 + γ )} {( W / H )}^{( 1 - γ )}}{{D}_{x}^{γ}} ] + b \} \frac{\partial C}{\partial y} \} + {F ( k )}_{s , i} C - S = 0 ;$$
$${F ( k )}_{s , i} = F ( C , {DO}_{s} , {DO}_{i} , Q , t , Δ x , Δ y ) ;$$
wherein F(k)_{s,i} is a pollutant attenuation coefficient under the fourth preset environmental condition.

2. The refined calculation method for total pollutant discharge under the complex river condition according to claim 1, wherein separately calculating the water environmental capacity for the pollutant in the studied river reach during the low-flow period and the high-flow period is represented by a formula:
$$w = {Q}_{0} ( {C}_{s} - {C}_{0} ) + {KVC}_{s} ,$$
wherein Q₀ is inflow discharge at an inlet cross-section, Cₛ is a water quality standard for a water body, C₀ is a water quality concentration at the inlet cross-section, K is a pollutant attenuation coefficient, and V is a water body volume.
